(19) United States
(12) Patent Application Publication
(10) Pub. No.: US 2024/0006060 A1
(43) Pub. Date: Jan. 4, 2024

(54) MACHINE LEARNING BASED SYSTEMS AND METHODS FOR CLASSIFYING ELECTRONIC DATA AND GENERATING MESSAGES

(71) Applicant: Centene Corporation, Saint Louis, MO (US)

(72) Inventors: Janet G. Behlmann, Wentzville, MO (US); Brian Dean Margherio, Edwardsville, IL (US); Adam Richardson, O'Fallon, MO (US); Kyle B. Schenthal, Clayton, MO (US)

(21) Appl. No.: 18/344,683

(22) Filed: Jun. 29, 2023

Related U.S. Application Data
(60) Provisional application No. 63/357,335, filed on Jun. 30, 2022.

(51) Int. Cl. G16H 40/20; G16H 50/30

(52) U.S. Cl. CPC G16H 40/20; G16H 50/30

(57) ABSTRACT

Described herein are an intelligent classification (IC) computing system including at least one processor in communication with at least one database, a non-transitory computer-readable storage medium having computer-executable instructions embodied thereon that are executable by the IC computing system, and a method implemented using the IC computing system. The at least one processor is configured to receive a message including admission data associated with at least one patient and configure the admission data into input data for a machine learning (ML) model configured to automatically classify the data by admission type, to input the input data into the ML model and based upon an output from the ML model, determine an admission type associated with the at least one patient, and to generate an authorization message associated with the at least one patient and transmit the authorization message to an external computing device for approval.

### CROSS-REFERENCE TO RELATED APPLICATIONS

This application claims priority to U.S. Provisional Application Ser. No. 63/357,335, filed on Jun. 30, 2022, the content of which is hereby incorporated by reference in its entirety.

### BACKGROUND

The field of this disclosure relates generally to using machine learning to classify electronic data and generate messages and, more specifically, to network-based systems and methods that use machine learning for improved efficiency and accuracy in classifying electronic data and generating messages.

Many variables need to be considered in order to automatically classify data and generate messages. For example, when classifying data, certain factors may be known and certain factors may be unknown. Different entities may provide different portions of data and/or data in different formats. Data being classified incorrectly results in wasted resources being applied to data that belongs in a different category.

As an example, currently, when a patient visits a hospital, a managed care entity (MCE) may receive an authorization request via an admission, discharge, transfer (ADT) message. The authorization request provides insight for why admission is necessary. It includes diagnosis codes and assessment codes. The ADT message follows a certain messaging protocol wherein a limited amount of information about the reason for the hospital visit is actually provided to the MCE. Thus, the authorization request review process is actually initiated prior to the MCE being truly notified about whether the patient is being admitted to the hospital and to which department. The MCE may not be truly notified of the reason for the hospital visit for several more hours or even days later. Consequently, authorization teams (e.g., referral specialists and clinicians) at the MCEs waste considerable resources on requests that, if they were able to classify them correctly, may be automatically approved by the MCE. The workflow for certain authorizations differs depending upon the reason for the authorization or hospital visit (e.g., when an MCE is notified of a patient's admission to a hospital, a medical authorization may be created based on one possible reason for the visit that is subsequently voided only to create a new authorization for a different reason for the visit). For example, when a patient is admitted to a hospital, an ADT message is generated and transmitted with certain limited information.

From that limited information, an MCE may have to determine whether to classify the visit as relating to obstetrics (OB), behavioral health (BH), or general medical (GM) to just name a few categories. BH authorizations may have a workflow that is different from OB or General Medical, so mislabeling/misclassifying data results in authorization teams wastes resources on requests that belong to a different category. It is important to classify the visit to the proper category as quickly as possible in order to obtain proper authorizations and approval for coverage.

Currently, in most hospital/MCE environments, a manual process is performed in order to classify authorizations. For example, an authorization request is transmitted via fax, phone, or email from a hospital to an MCE. A referral specialist then manually verifies the patient eligibility with the MCE and has to manually create an authorization. The referral specialist then has to verify if the hospital is a participating provider (PAR) or a non-participating provider (Non-PAR) with respect to the MCE. All of these manual steps are eliminated by the systems and methods described herein.

The systems and methods described herein are configured to accurately predict why a patient is being admitted to a hospital on a particular visit and thus, outputs fewer voided authorizations and increases productivity of authorization teams. Overall, accurate authorization predictions will decrease MCE expenses and improve the MCE relationships with its health plans. Thus, more sophisticated and automated systems and methods are desired for automatically classifying data and generating messages.

### BRIEF DESCRIPTION

An intelligent classification (IC) computing system including at least one processor in communication with at least one database is described herein. The at least one processor is configured to receive a message including admission data associated with at least one patient and configure the admission data into input data for a machine learning (ML) model configured to automatically classify the data by admission type. The at least one processor is also configured to input the input data into the ML model and based upon an output from the ML model, determine an admission type, of a plurality of admission types, associated with the at least one patient. The at least one processor is further configured to generate an authorization message associated with the at least one patient and transmit the authorization message to an external computing device for approval.

A non-transitory computer-readable storage medium having computer-executable instructions embodied thereon is also described herein. When executed by an intelligent classification (IC) computing system including at least one processor in communication with at least one database, the computer-readable instructions cause the IC computing system to receive a message including admission data associated with at least one patient. The computer-readable instructions also cause the IC computing system to configure the admission data into input data for a machine learning (ML) model configured to automatically classify the data by admission type. The computer-readable instructions also cause the IC computing system to input the input data into the ML model. The computer-readable instructions also cause the IC computing system to, based upon an output from the ML model, determine an admission type, of a plurality of admission types, associated with the at least one patient. The computer-readable instructions also cause the IC computing system to generate an authorization message associated with the at least one patient. The computer-readable instructions also cause the IC computing system to transmit the authorization message to an external computing device for approval.

A method implemented using an intelligent classification (IC) computing system including at least one database communicatively coupled to a processor is also described herein. The method includes receiving a message including admission data associated with at least one patient. The method also includes configuring the admission data into input data for a machine learning (ML) model configured to automatically classify the data by admission type. The method also includes inputting the input data into the ML model. The method also includes, based upon an output from the ML model, determining an admission type, of a plurality of admission types, associated with the at least one patient. The method also includes generating an authorization message associated with the at least one patient. The method also includes transmitting the authorization message to an external computing device for approval.

### BRIEF DESCRIPTION OF THE DRAWINGS

FIGS. 1-6 show example embodiments of the systems and methods described herein.

FIG. 1 is a schematic diagram illustrating an example intelligent classification (IC) computing system for automatic classification of data and generation of messages in accordance with the present disclosure.

FIG. 2A is an example partial prior art flow diagram including manual classification of data and generation of messages.

FIG. 2B is an example flow diagram for automatic classification of data and generation of messages in accordance with the present disclosure.

FIG. 3A and FIG. 3B illustrate example results of automatic classification of data and generation of messages in accordance with the present disclosure.

FIG. 4 is an example configuration of a computing device in accordance with the present disclosure.

FIG. 5 is an example configuration of server components for automatic classification of data and generation of messages in accordance with the present disclosure.

FIG. 6 is an example method for automatic classification of data and generation of messages in accordance with the present disclosure.

### DETAILED DESCRIPTION

The following detailed description illustrates embodiments of the disclosure by way of example and not by way of limitation. The description enables one skilled in the art to make and use the disclosure. It also describes several embodiments, adaptations, variations, alternatives, and uses of the disclosure, including what is presently believed to be the best mode of carrying out the disclosure.

In the example embodiment, an intelligent classification (IC) computing device is described. The IC computing device is configured to create a multiclass classification machine learning (ML) model/algorithm that includes classifiers for at least classes “OB” (e.g., obstetrics), “BH” (e.g., behavioral health) and “Medical” (e.g., other medical visit). The ML model is configured to receive a message from a healthcare provider in response to a visit by a patient to the healthcare provider, and classify the message (which in turn classifies the visit) into one of the classes predefined in the system. The ML model may be configured to return classification probabilities for each of these messages, thereby indicating a percentage of likelihood that a particular visit to the healthcare provider is for OB, BH, or other Medical reasons. This provides a significant improvement over known methodologies for classifying such patient visits. These improvements over known methodologies are described below in more detail. One such improvement includes the ability to calculate a classification probability for a visit, which is not done under the known systems.

The class predicted by the model (e.g., from data in an admission, discharge, transfer (ADT) message and other data) will determine the workflow for the associated visit. For example, the IC computing device may automatically generate an authorization form based upon the output from the ML model. Classification changes within a visit (e.g., Message 1 is predicted to be OB and Message 2 is Medical) can flag that a potential misclassification was made. If classification probabilities are obtained, a measure of doubt can be added that flags a message for human review and/or is used to further train the ML model.

Inputs to the model may include data from ADT messages as well as patient demographic fields and Notification of Pregnancy (NOP) assessment fields. Data in Notification of Pregnancy (NOP) assessment fields are particularly useful in automatic classification. A representational state transfer application program interface (RESTful API) may be used as a user interface for the model. Messages come in one at a time and require retrieving additional patient data from patient databases. A gradient boosting ensemble of decision trees is used as a proof of concept (POC) due to its robustness and ease of development. Features include select ADT message fields previously parsed manually along with patient data.

An API is implemented for the ML model using an API package (e.g., FastAPI Python package) with endpoints for API description, health check, and prediction. The model with the API may be containerized (e.g., with standalone, executable packages of software such as one or more Docker containers). The container is deployed locally with successful API interaction from outside the container. In some embodiments, the API may require the user to parse the ADT message and retrieve all the required patient, plan, and NOP data from provider databases. In some embodiments, ADT messages may automatically be used as the input via separate and/or combined ADT parsing API. This gives the system greater control over the data and allows the model to use additional features that have not been extracted using current ADT message parsers. Having an ADT parsing API as a separate service will allow more easy leverage of ADT data (e.g., in classifying behavior health vs. medical authorizations).

For example, data inputted to prior models may have been limited to state, facility, age, sex, health plan, diagnosis code, visit duration, product (e.g., health coverage benefits using a particular network such as health maintenance organization, preferred provider organization, exclusive provider organization, point of service, and/or indemnity), and/or expected due date. In other words, prior model inputs may have been limited to certain data pulled from an ADT message (e.g., the inputs listed above may all be included in an ADT message, other than expected due date).

Notably, the model described herein utilizes factors unknown to previous models that improve classification, as described herein. For example, the model pulls information from ADT messages as described above, in addition to information from other sources. In the example embodiment, factors utilized by the model include state, facility, age, sex, health plan, diagnosis (dx) code, visit duration, product, expected due date—in addition to inpatient future risk, risk score, inpatient stay probability, er risk score, nest score, ADT type, trimester 1 visit date, trimester 2 visit date, trimester 3 visit date, and Clinical Classifications Software Refined (CCSR—e.g., as explained at the following address https://www.hcup-us.ahrq.gov/toolssoftware/ccsr/ccs_refined.jsp).

The risk scores and inpatient stay probability described above may be internal measures created by data science teams. Nest score may be measure of socioeconomic status. By utilizing the additional information, including trimester visit dates, the model has achieved improved performance in predicting OB classifications. Further, risk scores and nest scores have helped the model achieve improved performance in at least predicting BH classifications. Utilization of inpatient and ER risk scores have helped the model achieve improved performance in all classifications described herein (e.g., OB, BH, Medical, etc.).

A processor or a processing element may be trained using supervised or unsupervised machine learning, and the machine learning program may employ a neural network, which may be a convolutional neural network, a deep learning neural network, or a combined learning module or program that learns in two or more fields or areas of interest. Machine learning may involve identifying and recognizing patterns in existing data in order to facilitate making predictions for subsequent data. Models may be created based upon example inputs in order to make valid and reliable predictions for novel inputs.

Additionally or alternatively, the machine learning programs may be trained by inputting sample data sets or certain data into the programs, such as images, statistics and information, and/or historical data. For example, the model described herein may classify patient visits based upon the described inputs/training. The model may then be refined and improved by looping the classifications (e.g., and any identified accuracies and/or inaccuracies in the model output) back in to train the model.

The machine learning programs may utilize deep learning algorithms that may be primarily focused on pattern recognition, and may be trained after processing multiple examples. The machine learning programs may include Bayesian program learning (BPL), voice recognition and synthesis, image or object recognition, optical character recognition, and/or natural language processing—either individually or in combination. The machine learning programs may also include natural language processing, semantic analysis, automatic reasoning, and/or other types of machine learning or artificial intelligence.

In supervised machine learning, a processing element may be provided with example inputs and their associated outputs, and may seek to discover a general rule that maps inputs to outputs, so that when subsequent novel inputs are provided the processing element may, based upon the discovered rule, accurately predict the correct output. In unsupervised machine learning, the processing element may be required to find its own structure in unlabeled example inputs.

As described above, the systems and methods described herein may use machine learning, for example, for pattern recognition. That is, machine learning algorithms may be used by the IC computing system to automatically classify data and generate messages. The machine learning algorithms may also be used by the IC computing system to attempt to predict why a patient is being admitted to a hospital on a particular visit. Accordingly, the systems and methods described herein may use machine learning algorithms for both pattern recognition and predictive modeling. As an example, the IC computing system may utilize machine learning techniques when automatically classifying data, predicting an authorization, and generating messages, as described herein.

For reference, some hospitals have a large proportion of OB authorizations. Because authorization review resources are more likely to be wasted at these hospitals, and contacting these hospitals regarding such requests risks hospital-health plan relations, today, an MCE may automatically approve requests from hospitals that are deemed to have disproportionally large OB departments (e.g., OB admits are unique as virtually all authorization requests related to delivery may be automatically approved by a provider). This automatic process increases operating costs by approving authorizations that a provider may otherwise deny—and is no longer necessary with implementation of the IC computing device as described herein.

Being able to successfully predict authorizations from ADT admission messages when received by an MCE will remove the need for blanket approvals of designated hospitals, thereby reducing MCE expenses. Additionally, correctly categorizing authorizations from the beginning can allow referral specialists to more likely create an authorization correctly the first time. It is estimated that each incorrectly classified authorization costs on average $4 in lost labor and fees.

The embodiments described herein provide substantial benefits to MCEs with little risk. Currently, referral specialists waste time evaluating authorization requests only to later learn they have been miscategorized. Risks occur with misclassifications in known practices. As examples, in the known practices, false negatives include a failure to correctly classify an authorization and false positives include mislabeling an authorization that results in a delayed approval.

At least some of the technical problems addressed by this system include: i) data being misclassified; ii) resources being wasted because of resource allocation/application to misclassified data; iii) inefficiencies in current manual processes/workflows for classifying data; and iv) high costs associated with current manual processes/steps in classification of data.

A technical effect of the systems and methods described herein is achieved by performing at least one of: i) receiving a message including admission data associated with at least one patient; ii) configuring the admission data into input data for a machine learning (ML) model configured to automatically classify the data by admission type; iii) inputting the input data into the ML model; iv) based upon an output from the ML model, determining an admission type, of a plurality of admission types, associated with the at least one patient; generating an authorization message associated with the at least one patient; and v) transmitting the authorization message to an external computing device for approval.

The technical effects and advantages achieved by this system are at least one of: i) correctly classifying data; ii) saving resources by not applying resources to misclassified data; iii) more efficient workflows for classifying data; and iv) removing the high costs associated with current manual processes/steps in classification of data.

The systems and methods described herein may use machine learning to train and utilize the model, for example, using pattern recognition. That is, machine learning algorithms may be used by the IC computing system to automatically classify data and generate messages. The model may be trained using a plurality of data sets. For example, the model may be trained using historical ADT messages, namely the data variables contained within each historical ADT message. In addition, other historical data not contained in the ADT message may be used to further help classify a patient visit. In other words, an ADT message may include a patient's name which may be parsed by the system from the ADT message. The patient name may then be used by the system to retrieve other data about the patient that the system may have access to. This other data may be further used as an input into the model to help further classify the particular visit by the patient.

Further, the other historical data not contained in the ADT message may be analyzed and certain data (e.g., all of the other historical data or the most important of the other historical data for classification, as determined by the ML model (via training and re-training)) may be automatically requested. For example, the ML model may identify at least a portion of desired data not included in the ADT message (e.g., that may improve performance of the ML model for a particular classification) and generate and transmit a request for at least the portion of desired data (e.g., to a device associated with a hospital, a patient, etc.). Upon receipt of the requested data, the requested data may be utilized by the ML model along with the other data described herein. In some embodiments, the ML model may wait a predetermined amount of time upon sending the request(s) before generating an output (e.g., a few hours, to provide time for a response before predicting a classification).

In another example, certain medical facilities (e.g., hospitals or medical providers) may send ADT messages having patterns of data for particular reasons for a visit. For example, Hospital A may send ADT messages with certain data variables provided and included therein for an OB visit. The system described herein is able to detect these patterns of data within messages sent by Hospital A such that when a later ADT message is received from Hospital A, the system/model is able to recognize the pattern of data variables provided within the ADT message as being associated with a particular classification of visit. Thus, the system is able to parse the new data received and accurately classify the visit.

In one embodiment, a computer program is provided, and the program is embodied on a computer-readable medium. In an example embodiment, the system is executed on a single computer system, without requiring a connection to a server computer. In a further example embodiment, the system is run in a Windows® environment (Windows is a registered trademark of Microsoft Corporation, Redmond, Washington). In yet another embodiment, the system is run on a mainframe environment and a UNIX® server environment (UNIX is a registered trademark of X/Open Company Limited located in Reading, Berkshire, United Kingdom). In a further embodiment, the system is run on an iOS® environment (iOS is a registered trademark of Apple Inc. located in Cupertino, CA). In yet a further embodiment, the system is run on a Mac OS® environment (Mac OS is a registered trademark of Apple Inc. located in Cupertino, CA). In a further embodiment, the system is run on a Linux® environment (Linux is a registered trademark of Linus Torvalds in the United States and other countries). The application is flexible and designed to run in various different environments without compromising any major functionality. In some embodiments, the system includes multiple components distributed among a plurality of computing devices. One or more components are in the form of computer-executable instructions embodied in a computer-readable medium. The systems and processes are not limited to the specific embodiments described herein. In addition, components of each system and each process can be practiced independently and separately from other components and processes described herein. Each component and process can also be used in combination with other assembly packages and processes.

In one embodiment, a computer program is provided, and the program is embodied on a computer-readable medium and utilizes a Structured Query Language (SQL) with a client user interface front-end for administration and a web interface for standard user input and reports. In another embodiment, the system is web enabled and is run on a business entity intranet. In yet another embodiment, the system is fully accessed by individuals having an authorized access outside the firewall of the business-entity through the Internet. In a further embodiment, the system is being run in a Windows® environment (Windows is a registered trademark of Microsoft Corporation, Redmond, Washington). The application is flexible and designed to run in various different environments without compromising any major functionality.

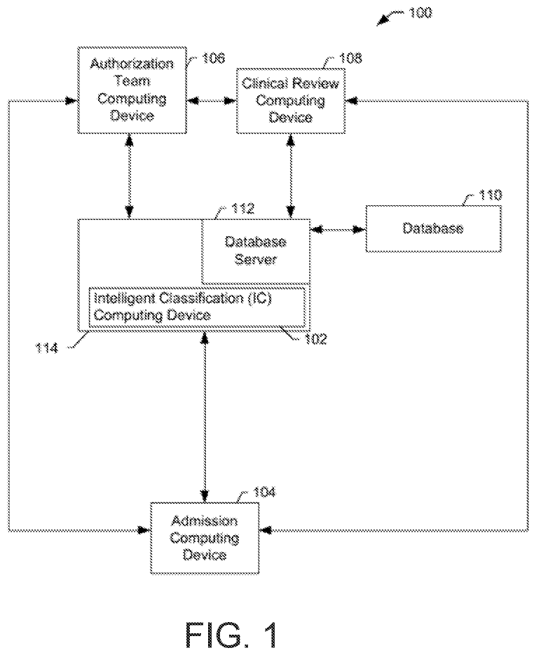

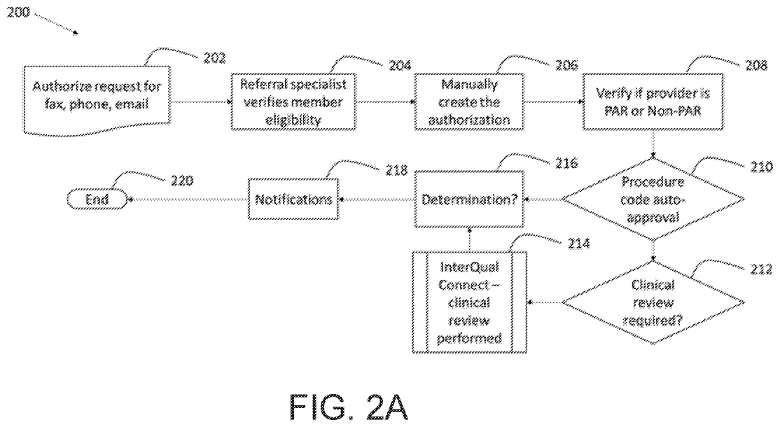

FIG. 1 is a schematic diagram illustrating an example intelligent classification (IC) computing system 100 for automatic classification of data and generation of messages in accordance with the present disclosure. IC computing system 100 includes at least one IC computing device 102, (e.g., implemented on a server 114), and in communication with at least one admission computing device 104, at least one authorization team computing device 106, and/or at least one clinical review computing device 108. Although FIG. 1 only shows one of each computing device 102-108, it should be recognized that any number of computing devices 102-108 may be used in IC computing system 100.

AC computing device 102 is further in communication with at least one database 110 that may store and/or process data, such as ADT data, patient data (e.g., a patient name, a patient home address, a patient phone number, a patient email address, patient medical information, a patient-preferred language, other patient preferences, and/or other data associated with a patient), and/or any other data described herein. A database server 112 may be in communication with database 110, which contains information on a variety of matters (e.g., patient data), as described herein in greater detail. In one embodiment, database 110 is stored on server 114 and may be accessed by logging onto server 114 and/or IC computing device 102 via, for example, authorization team computing device 106. In another embodiment, database 110 is stored remotely from server 114 and may be non-centralized.

In the example embodiment, IC computing device 102 is configured to receive data from admission computing device 104, for example, regarding a patient admission at a hospital associated with device 104. Device 102 is configured to analyze the data received from device 104. In the example embodiment, device 102 utilizes data (e.g., ADT data) received from device 104 by using and/or configuring the received data as inputs for a machine learning (ML) model implemented by device 102.

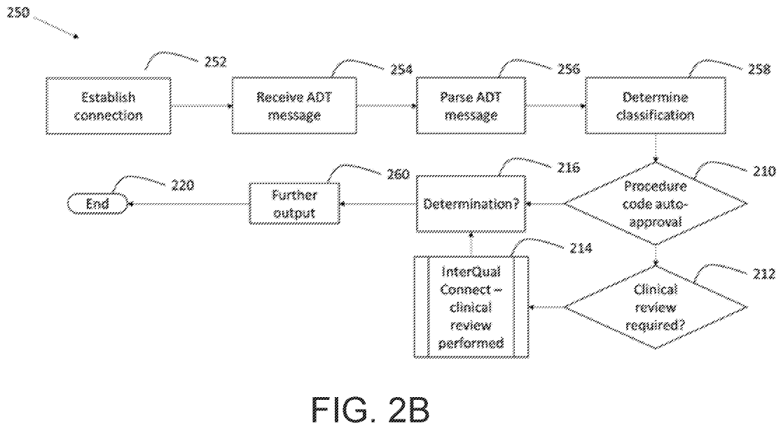

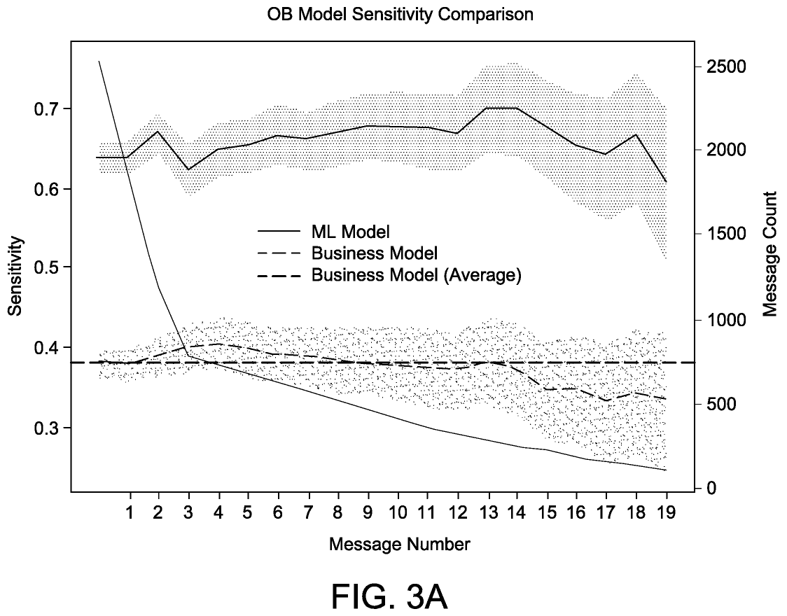

Outputs of the ML model may then be automatically implemented and/or transmitted to authorization team computing device 106 (e.g., a smartphone, laptop, tablet, etc.) and/or clinical review computing device 108 (e.g., a smartphone, laptop, tablet, etc.). Data may also be transmitted between device 106 and device 108 (e.g., as described below with respect to FIG. 2).

In some embodiments, outputs of the ML model cause an interface to be displayed at at least one of devices 104-108 for review. For example, the output may include an authorization with fillable fields (e.g., some fields may be automatically filled by the ML model, and some fields may allow for user input). In some embodiments, device 102 may cause an interface to be displayed at at least one of devices 104-108 before patient data is inputted to the ML model. For example, in some embodiments an interface may be displayed that allows for the user to select and/or modify data that will be inputted to the ML model (e.g., before classification of a particular patient visit).

In the example embodiment, IC computing device 102 may create a multiclass classification machine learning (ML) model algorithm with at least classes “OB” (e.g., obstetrics), “BH” (e.g., behavioral health) and “Medical” (e.g., other Medical visit). The ML model may also return classification probabilities (e.g., providing a significant improvement over known models). In other words, additionally/alternatively, the ML model may provide percentage probabilities of a certain OB, BH, and or Medical classification for a patient admission. The class predicted by the ML model (e.g., from data in an automated discharge transfer (ADT) message and other data) may determine the workflow for the associated visit. Classification changes within a visit (e.g., Message 1 is predicted to be OB and Message 2 is Medical) can flag (e.g., as detected by device 102) that a potential misclassification was made. If classification probabilities are obtained, a measure of doubt can be added that flags a message for human review.

Inputs to the model may include data from ADT messages as well as patient demographic fields and Notification of Pregnancy (NOP) assessment fields (e.g., as transmitted from device 104). A representational state transfer application program interface (RESTful API) may be used as a user interface for the model. Messages come in one at a time and require retrieving additional patient data from patient databases (e.g., database 110).

Notably, IC computing device 102 utilizes factors unknown to previous models that improve classification, as described herein. For example, IC computing device 102 pulls information from ADT messages (e.g., from device 104), in addition to information from other sources (e.g., database 110). In the example embodiment, factors utilized by the model include state, facility, age (e.g., patient age (e.g., in seconds)), sex (e.g., gender of patient), health plan (e.g., health plan code member code such as FL, LA, OH, TX, etc.), diagnosis (dx) code (e.g., ICD10 diagnosis code from ADT message), visit duration (e.g., time difference between admission date and discharge date in seconds), product (e.g., health plan name in detail from ADT message), expected due date—in addition to inpatient future risk (e.g., the relative risk of the member for the next 12 months compared to other plan members with respect to inpatient stays), risk score (e.g., the relative risk of the member for the next 12 months compared to other plan members with respect to total cost), inpatient stay probability, er risk score (e.g., the likelihood that the member will have 1 or more ER visits in the next 12 months), nest score, engagement score (e.g., the likelihood that the member will successfully engage in future care management activities), ADT type (e.g., ADT message type such a01, a06), trimester 1 visit date (e.g., time difference between first trimester and admission date), trimester 2 visit date (e.g., time difference between second trimester and admission date), trimester 3 visit date (e.g., time difference between third trimester and admission date), exp diff (e.g., time difference between expected due date and admission date), Clinical Classifications Software Refined (CCSR—e.g., aggregates ICD10 diagnosis codes into clinically meaningful categories across body system—see also the following address https://www.hcup-us.ahrq.gov/toolssoftware/ccsr/ccs_refined.jsp), plan type (e.g., Insurance plan type such as Medicare, Medicaid, Commercial, etc.), source (e.g., internal code for healthplan), total BH risk score (e.g., relative total risk of future behavioral health claims over next 12 months, weighed sum of IP, OP, ER, PCP, SPC, OM, Rx), er risk score fc (e.g., relative risk of future emergency room claims over next 12 months (excluding bh)), ip risk score (e.g., relative risk of future inpatient claims over next 12 months(excluding bh), and total risk score (e.g., relative total risk of future claims over next 12 months (excluding bh), weighed sum of IP, OP, ER, PCP, SPC, OM, Rx).

The risk scores and inpatient stay probability may be internal measures created by internal data science teams that are stored in database 110. Nest score may be measure of socioeconomic status. By utilizing the additional information, including trimester visit dates, IC computing device 102 and the ML model achieve improved performance in predicting OB classifications. Further, risk scores and nest scores have helped IC computing device 102 and the ML model achieve improved performance in at least prediction BH classifications. Utilization of inpatient and ER risk scores have helped IC computing device 102 and the ML model achieve improved performance in all classifications described herein (e.g., OB, BH, Medical, etc.).

In some embodiments, IC computing device 102 inputs certain factors into the ML model in order of importance and/or the ML model may weigh certain factors in order of importance. For example, in some embodiments, age, facility, sex, inpatient future risk, cars, diagnosis code may be weighted heavily with respect to other factors. In some embodiments, such as OB embodiments, age, sex, inpatient future risk, facility, cars, and diagnosis code may be weighted heavily with respect to other factors. In some embodiments, such as BH embodiments, facility, inpatient future risk, cars, age, health plan, adt type may be weighted heavily with respect to other factors.

A gradient boosting ensemble of decision trees is used as part of the ML model as a proof of concept (POC) due to its robustness and ease of development. The initial features include select ADT message fields previously parsed manually along with patient data.

In some embodiments, an API is implemented for the ML model using an API package (e.g., FastAPI Python package) with endpoints for API description, health check, and prediction. The model with the API may be containerized (e.g., with standalone, executable packages of software such as one or more Docker containers). The container may be deployed locally with successful API interaction from outside the container. The API may require a user to parse the ADT message and retrieve all the required patient, plan, and NOP data from provider databases (e.g., database 110). In some embodiments, ADT messages themselves are used as the input and a separate ADT parsing API may parse the message. This gives the ML model greater control over the data and allows the ML model to use additional features that are not extracted using a current ADT message parser. Having the ADT parsing API as a separate service will allow more efficient leverage of ADT data in the future (e.g., in classifying behavior health vs. medical authorizations).

FIG. 2A is an example partial prior art flow diagram 200 including manual classification of data and generation of messages.

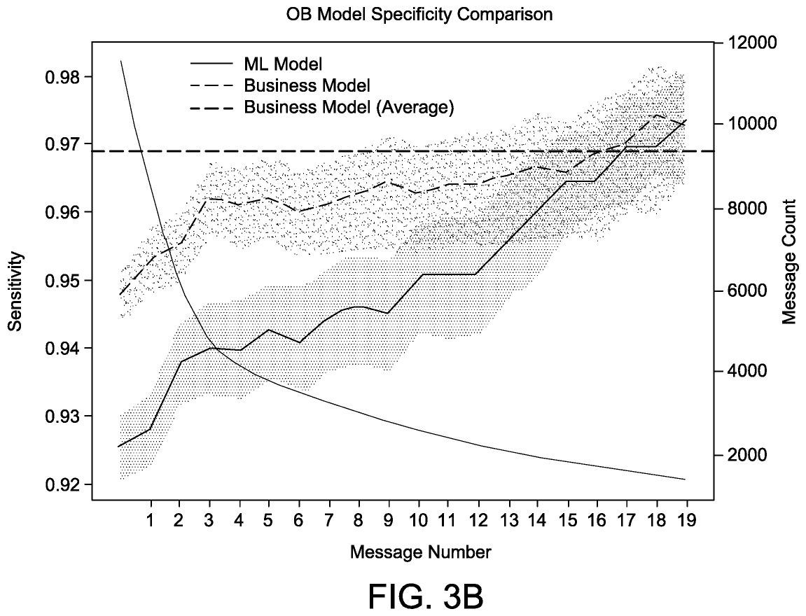

As shown in FIG. 2A, steps 202-208 include steps in known systems that are eliminated by implementing IC computing device 102. In known systems, a request is manually generated 202 for fax, phone, or email, a referral specialist manually verifies 204 patient eligibility, a team member (e.g., an authorization team member) manually creates 206 an authorization, and verifies 208 whether the MCE is a participating provider (PAR) or a non-participating provider (Non-PAR). As explained below with respect to FIGS. 2B, 3A, and 3B, device 102 eliminates the need for steps 202-208.

Steps 210-220 may still be required after implementation of device 102. In some embodiments, device 102 may automatically control steps 210-220. As described herein, a code may receive 210 auto-approval. Upon a code not being auto approved, it is determined 212 that clinical review is required and clinical review is performed 214. The outcome of either the review or the auto-approval is then determined 216. Consequently, notifications are generated according to the outcome and the notifications are transmitted 218 to appropriate parties (e.g., providers, hospitals, etc.) before the flow ends 220.

FIG. 2B is an example flow diagram 250 for automatic classification of data and generation of messages in accordance with the present disclosure. For example, diagram 250 illustrates the implementation of device 102 to eliminate steps 202-208 and 218 from known processes.

In the example shown in FIG. 2B, a connection between IC computing device 102 and one or more example admission computing devices 104 is established 252 (e.g., thereby eliminating the need for fax, phone, or email communication shown in step 202). When a patient is admitted to the hospital, IC computing device 102 automatically receives 254 an ADT message from device 104 and parses 256 the ADT message for relevant data. Parsing 256 is performed by a data parser that is configured to recognize and extract data from the ADT message.

The parser is configured to analyze ADT messages using the ADT message protocols. For example, the ADT message may include a variety of data variables including, but not limited to, member ID, member name, member birth date, member gender, date of the message, message type, admit location, admit type, admit date/time, admit reason, accommodation ID or code, accommodation text, hospital service, diagnosis code, diagnosis description, insurance company name, and/or discharge disposition. In some cases, the ADT message may include all or just some of these data variables. The parser is able to recognize this data and extract it, assigning the received data to the proper data fields for further analysis.

Device 102 then utilizes data from the ADT message with other data known by device 104 (e.g., patient data stored in database 110) to determine 258 a classification (e.g., OB, BH, Medical) for the visit, or a percentage of likelihood of a classification for the visit (e.g., thus eliminating the need for manually performing steps 204-210, above). Thus, determine step 258 is performed using a machine learning model that is configured or trained to recognize patterns of data included in the ADT message and other data that may be retrieved outside of the ADT message. The model is able to accurately classify the visit of the patient based upon the data inputs from the ADT message and the other data.

Device 102 may then automatically perform steps 210, 212, 214, 216, 260, and 220 depending on the classification output and/or percentage of likelihood of classification output. For example, device 102 may then determine whether the visit should receive 210 auto-approval or determine 212 that clinical review is required and perform/transmit a request for performance 214 of a clinical review. The outcome of either the review or the auto-approval is then determined 216. Consequently, further output 260 is produced according to the classification outcome. The further output 260 could include notifications that are automatically generated according to the outcome and then transmitted to appropriate parties (e.g., MCEs, hospitals, patients etc.) before the flow ends 220. The further output 260 could alternatively or additionally include a further output resulting from the classification outcome.

Generally, the classification outcome for a visit may result in any suitable further output known in the art that facilitates the system and method described herein. In some embodiments, the further output resulting from the classification outcome includes a data file. For example, a data file may be created once a visit is classified. The data file may be stored in a memory of a processor.

In some embodiments, at least one rule stored in the memory of the processor is applied to visit data in the data file once the visit is classified. The at least one rule applied to visit data may determine appropriate further output resulting from the classification output. The appropriate further output may also be stored in the data file. The at least one rule may originate from the ML model.

The data file may include visit data, classification data, and/or further output related to classification. The data file may include data including or relating to or automatically causing certain steps to be performed by the ML model including creating a form for a visit, creating an authorization form for a visit, predicting an estimated end date for a visit, scheduling a pre-discharge event, scheduling a post-discharge event, scheduling a home visit, scheduling a communication (e.g., text, pop-up, email, and/or telephone call) with a patient and/or healthcare provider and/or caregiver relating to a visit, scheduling a confirmation communication (e.g., text, pop-up, email, and/or telephone call) with a patient and/or healthcare provider and/or caregiver relating to a visit, making a communication with a patient and/or healthcare provider and/or caregiver relating to a visit, making a request to a patient and/or healthcare provider and/or caregiver for more information, predicting a cost estimate, scheduling a medication, predicting a medication, predicting follow-ups, predicting a spoken and/or written language of the patient, a health condition of the patient, a health condition of the baby, patient progress, tracking patient progress, predicting a treatment, and/or predicting future treatment. Further, the ML model may automatically identify parties involved with the above steps (e.g., patient, doctor, caregiver, hospital, pharmacy, etc.) and automatically transmit notifications associated with the steps to the appropriate parties (e.g., a notification for an automatically scheduled doctor visit may be transmitted to computer devices associated with the appropriate patient and doctor's office).

For example, in OB embodiments, the ML model may classify the visit as an OB visit (e.g., birth of a baby), and this classification may be stored in the data file. The classification may also be used to predict a treatment or relevant consideration, including but not limited to, natural childbirth or caesarean section, estimated end date for a visit, prediction of necessary home visits and follow-ups, assignment of a home healthcare professional, scheduling of home visits, and communication with the patient and home healthcare professional.

Certain model outputs may be overridden (e.g., see the certain steps listed above), for example if part of an output is determined not to be necessary or desired. Accordingly, the ML model may be re-trained based on detected overrides to improve future performance of the ML model.

FIG. 3A illustrates example results of automatic classification of data and generation of messages, as implemented using IC computing system 100 shown in FIG. 1, as compared to other systems with respect to sensitivity (e.g., the ability to correctly classify patient visits). FIG. 3B illustrates example results of automatic classification of data and generation of messages, as implemented using IC computing system 100 shown in FIG. 1, as compared to other systems with respect to specificity (e.g., the ability to not incorrectly classify patient visits).

The example system shown in comparison with system 100 is a prior model (e.g., and a prior model average). In some embodiments regarding OB admissions, the prior model represents a model including only use of 3 features: sex, age, and difference between admit date and due date (dt). The prior model may be based on intuition with no guarantees of local optimality and little/no support for threshold decisions. For example, the prior model may treat all ages between 15 and 55 equally even though the distribution of maternal ages at delivery is not uniform (e.g., very few deliveries by mothers over 40).

Similarly, with respect to the prior model, all visits within 2 weeks may be treated equally, but distribution of gestational age at birth is not symmetric and is skewed with fewer pregnancies extending beyond the expected due date than terminating prior to the due date. From an export of parsed ADT messages, it was found that almost no OB visits contained patients with maternal age greater than 45, and the 2-week time threshold missed approximately 22% of OB messages. The machine learning (ML) model described herein incorporates the same data and more (e.g., data from an ADT message and previously-known data regarding the patient) to improve prediction accuracy (by learning discriminants that minimize error). Providing additional relevant data to the ML model will further improve performance.

Similarly, up until now, MCEs may also use a prior model composed entirely of logical conjunctions to classify whether an authorization was BH. This prior model may only use one feature: diagnosis code Similar to the prior logic for OB, this prior model may be based on intuition. In addition, from previous analysis of ADT messages, it was found that around 80% lack diagnosis codes, meaning that many BH authorizations are missed by this prior logic.

While alternative approaches were considered (e.g., a rule-based approach, a single decision tree, a rule-based Repeated Incremental Pruning to Produce Error Reduction (RIPPER) algorithm, etc.), the quick, robust gradient boosting decision tree ensemble model was ultimately chosen for the example embodiment of the ML model described herein (and implemented by device 102).

Currently, only a few select fields from ADT messages are being parsed by MCEs. Notably, device 102 is configured to extract data (e.g., more than the few select fields) from the raw ADT messages (e.g., using an hl7 Python package).

The ML model's performance may be primarily focused on two metrics: sensitivity (accuracy of the individual classes) and total message misclassifications (0-1 loss). Additionally, these metrics have been analyzed over the course of their respective visits in creating the ML model by comparing performance of the different messages in the visits (e.g., the ML model should classify the visit consistently across messages). Elucidation of the model's performance by visit message index is critical for the model to deliver as it is more valuable to get the first message of a visit correct than the last. Performance is compared to that of known models, as described herein.

Model performance was measured on the test partition of the dataset after grouping the messages into visits (defined by patient ID and admit date) and using an 80-20 split on the collection of visits. Splitting on visits instead of messages is important to reduce overfitting that would result from training and testing messages from the same visit. Model generalizability and performance variance may be measured using stratified 10-fold cross-validation of the training partition (e.g., using np.array_split on the shuffled indices).

In overall misclassifications, the ML model has outperformed the known model with a 21.5% reduction in misclassifications. The gain in performance can be attributed at least in part to better performance with respect to OB messages as ML model sensitivity was 70% for the test dataset compared to the prior model with 39.1%. For BH messages, the model correctly predicted approximately 50% in the test set compared to 11% using the known model.

The known model's average test sensitivity is 37.8% determined using the entire dataset. This gain in sensitivity comes at the cost of a slight loss in specificity with the prior model performing at approximately 96% while the ML model performed at approximately 95% (e.g., the prior model seems to be filtering too many potentially OB visits due to a 2-week filter and the NOP requirement).

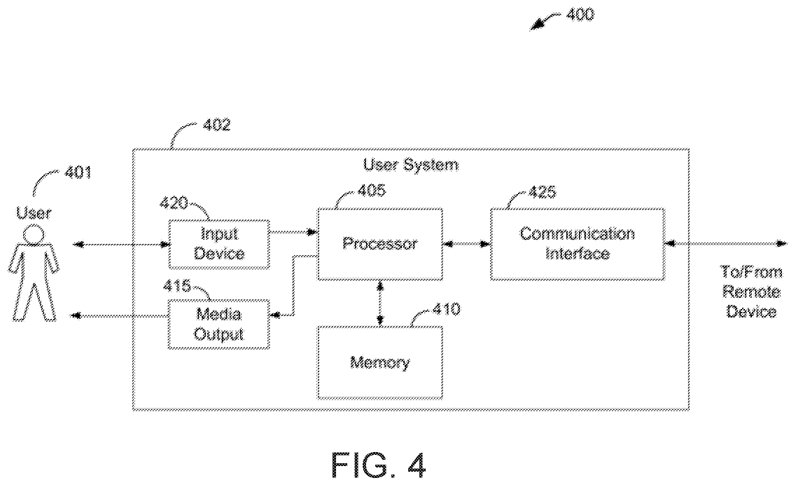

FIG. 4 illustrates an example configuration of a user system 402 operated by a user 401. In the example embodiment, user system 402 may be used to implement device 104, device 106, and/or device 108 (shown in FIG. 1), and may be used by user 401 to interact with IC computing device 102 (also shown in FIG. 1). In the example embodiment, user system 402 includes a processor 405 for executing instructions. In some embodiments, executable instructions are stored in a memory area 410. Processor 405 may include one or more processing units, for example, a multi-core configuration. Memory area 410 is any device allowing information such as executable instructions and/or written works to be stored and retrieved. Memory area 410 may include one or more computer readable media.

User system 402 also includes at least one media output component 415 for presenting information to user 401. Media output component 415 is any component capable of conveying information to user 401. In some embodiments, media output component 415 includes an output adapter such as a video adapter and/or an audio adapter. An output adapter is operatively coupled to processor 405 and operatively couplable to an output device such as a display device, a liquid crystal display (LCD), organic light emitting diode (OLED) display, or “electronic ink” display, or an audio output device, a speaker or headphones.

In some embodiments, user system 402 includes an input device 420 for receiving input from user 401. Input device 420 may include, for example, a keyboard, a pointing device, a mouse, a stylus, a touch sensitive panel, a touch pad, a touch screen, a gyroscope, an accelerometer, a position detector, or an audio input device. A single component such as a touch screen may function as both an output device of media output component 415 and input device 420. User system 402 may also include a communication interface 425, which is communicatively couplable to a remote device, such as IC computing device 102. Communication interface 425 may include, for example, a wired or wireless network adapter or a wireless data transceiver for use with a mobile phone network, Global System for Mobile communications (GSM), 3G, or other mobile data network or Worldwide Interoperability for Microwave Access (WIMAX).

Stored in memory area 410 are, for example, computer readable instructions for providing a user interface to user 401 via media output component 415 and, optionally, receiving and processing input from input device 420. A user interface may include, among other possibilities, a web browser and client application. Web browsers enable users, such as user 401, to display and interact with media and other information typically embedded on a web page or a website from IC computing device 102. A client application allows user 401 to interact with a server application from IC computing device 102.

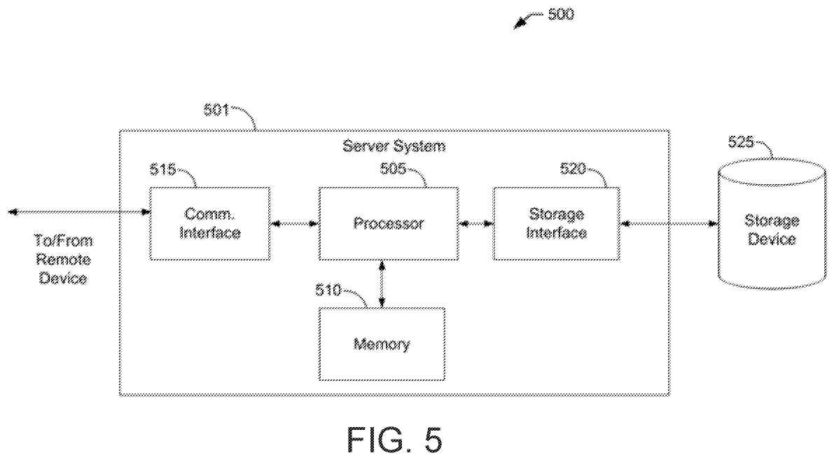

FIG. 5 illustrates an example configuration of a server system 501. Server system 501 may be used to implement IC computing device 102 (shown in FIG. 1), for example. Server system 501 includes a processor 505 for executing instructions. Instructions may be stored in a memory area 510, for example. Processor 505 may include one or more processing units (e.g., in a multi-core configuration) for executing instructions. The instructions may be executed within a variety of different operating systems on server system 501, such as UNIX, LINUX, Microsoft Windows®, etc. It should also be appreciated that upon initiation of a computer-based method, various instructions may be executed during initialization. Some operations may be required in order to perform one or more processes described herein, while other operations may be more general and/or specific to a particular programming language (e.g., C, C #, C++, Java, or other suitable programming languages, etc.).

Processor 505 is operatively coupled to a communication interface 515 such that server system 501 is capable of communicating with a remote device such as user system 402 (shown in FIG. 4) or another server system 501. For example, communication interface 515 may receive requests from, and send results to, device 104, device 106, and/or device 108 via the Internet, as illustrated in FIG. 1.

Processor 505 may also be operatively coupled to a storage device 525. For example, storage device 525 may be used to implement database 110 (shown in FIG. 1). Storage device 525 is any computer-operated hardware suitable for storing and/or retrieving data. In some embodiments, storage device 525 is integrated in server system 501. For example, server system 501 may include one or more hard disk drives as storage device 525. In other embodiments, storage device 525 is external to server system 501 and may be accessed by a plurality of server systems 501. For example, storage device 525 may include multiple storage units such as hard disks or solid state disks in a redundant array of inexpensive disks (RAID) configuration. Storage device 525 may include a storage area network (SAN) and/or a network attached storage (NAS) system.

In some embodiments, processor 505 is operatively coupled to storage device 525 via a storage interface 520. Storage interface 520 is any component capable of providing processor 505 with access to storage device 525. Storage interface 520 may include, for example, an Advanced Technology Attachment (ATA) adapter, a Serial ATA (SATA) adapter, a Small Computer System Interface (SCSI) adapter, a RAID controller, a SAN adapter, a network adapter, and/or any component providing processor 505 with access to storage device 525.

Memory area 510 may include, but are not limited to, random access memory (RAM) such as dynamic RAM (DRAM) or static RAM (SRAM), read-only memory (ROM), erasable programmable read-only memory (EPROM), electrically erasable programmable read-only memory (EEPROM), and non-volatile RAM (NVRAM). The above memory types are exemplary only, and are thus not limiting as to the types of memory usable for storage of a computer program.

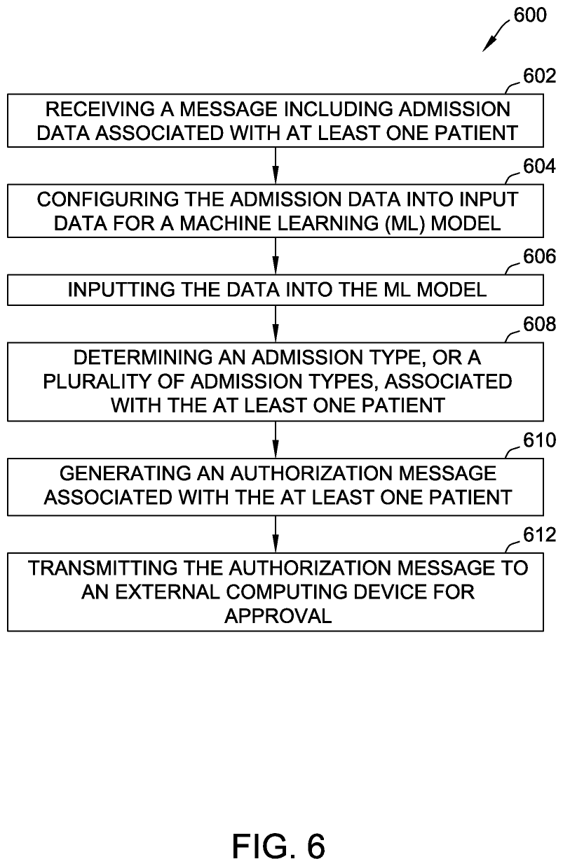

FIG. 6 is an example method 600 for automatic classification of data and generation of messages in accordance with the present disclosure. In the example embodiment, method 600 includes receiving 602 a message including admission data associated with at least one patient and configuring 604 the admission data into input data for a machine learning (ML) model configured to automatically classify the data by admission type. In some embodiments of method 600, the message includes an admission discharge transfer (ADT) message.

Also, method 600 includes inputting 606 the input data into the ML model and, based upon an output from the ML model, determining 608 an admission type, of a plurality of admission types, associated with the at least one patient. In some embodiments of method 600, the plurality of admission types includes at least one of an obstetrics (OB) type, a behavioral health (BH) type, or a Medical type. Further, method 600 includes generating 610 an authorization message associated with the at least one patient and transmitting 612 the authorization message to an external computing device for approval.

The computer-implemented methods discussed herein may include additional, less, or alternate actions, including those discussed elsewhere herein. The methods may be implemented via one or more local or remote processors, transceivers, servers, and/or via computer-executable instructions stored on non-transitory computer-readable media or medium.

Additionally, the computer systems discussed herein may include additional, less, or alternate functionality, including that discussed elsewhere herein. The computer systems discussed herein may include or be implemented via computer-executable instructions stored on non-transitory computer-readable media or medium.

A processor or a processing element may be trained using supervised or unsupervised machine learning, and the machine learning program may employ a neural network, which may be a convolutional neural network, a deep learning neural network, or a combined learning module or program that learns in two or more fields or areas of interest. Machine learning may involve identifying and recognizing patterns in existing data in order to facilitate making predictions for subsequent data. Models may be created based upon example inputs in order to make valid and reliable predictions for novel inputs.

Additionally or alternatively, the machine learning programs may be trained by inputting sample data sets or certain data into the programs, such as images, statistics and information, and/or historical data. The machine learning programs may utilize deep learning algorithms that may be primarily focused on pattern recognition, and may be trained after processing multiple examples. The machine learning programs may include Bayesian program learning (BPL), voice recognition and synthesis, image or object recognition, optical character recognition, and/or natural language processing—either individually or in combination. The machine learning programs may also include natural language processing, semantic analysis, automatic reasoning, and/or other types of machine learning or artificial intelligence.

In supervised machine learning, a processing element may be provided with example inputs and their associated outputs, and may seek to discover a general rule that maps inputs to outputs, so that when subsequent novel inputs are provided the processing element may, based upon the discovered rule, accurately predict the correct output. In unsupervised machine learning, the processing element may be required to find its own structure in unlabeled example inputs.

Exemplary embodiments of this disclosure include, but are not limited to the following:
Embodiment 1. An intelligent classification (IC) computing system comprising at least one processor in communication with at least one database, the at least one processor configured to:
receive a message including admission data associated with at least one patient;
configure the admission data into input data for a machine learning (ML) model configured to automatically classify the data by admission type;
input the input data into the ML model;
based upon an output from the ML model, determine an admission type, of a plurality of admission types, associated with the at least one patient;
generate an authorization message associated with the at least one patient; and
transmit the authorization message to an external computing device for approval.
Embodiment 2. The IC computing system of the preceding clause, wherein the message comprises an admission discharge transfer (ADT) message.
Embodiment 3. The IC computing system of any preceding clause, wherein the plurality of admission types comprises at least one admission type selected from the group consisting of an obstetrics (OB) type, a behavioral health (BH) type, and a Medical type.
Embodiment 4. The IC computing system of any preceding clause, wherein the ML model utilizes a factor selected from the group consisting of state, facility, age, sex, health plan, diagnosis (dx) code, visit duration, product, expected due date, inpatient future risk, risk score, inpatient stay probability, er risk score, nest score, ADT type, trimester 1 visit date, trimester 2 visit date, trimester 3 visit date, Clinical Classifications Software Refined, plan type, source, total BH risk score, er risk score fc, ip risk score, total risk score, and combinations thereof.
Embodiment 5. The IC computing system of any preceding clause, wherein the ML model utilizes a factor selected from the group consisting of trimester 1 visit date, trimester 2 visit date, trimester 3 visit date, and combinations thereof.
Embodiment 6. The IC computing system of any preceding clause, wherein the ML model utilizes a factor selected from the group consisting of age, facility, sex, inpatient future risk, cars, diagnosis code, health plan, adt type, and combinations thereof.
Embodiment 7. The IC computing system of any preceding clause, wherein the ML model employs a neural network selected from the group consisting of a convolutional neural network, a deep learning neural network, a combined learning module, a program that learns in two or more fields or areas of interest, and combinations thereof.
Embodiment 8. The IC computing system of any preceding clause, wherein the ML model is configured for pattern recognition and/or predictive modeling.
Embodiment 9. The IC computing system of any preceding clause, wherein the processor is further configured to produce a further output based on the automatic classification of the data.
Embodiment 10. The IC computing system of any preceding clause, wherein the processor is further configured to create a data file based on the automatic classification of the data.
Embodiment 11. A non-transitory computer-readable storage medium having computer-executable instructions embodied thereon, wherein when executed by an intelligent classification (IC) computing system including at least one processor in communication with at least one database, the computer-readable instructions cause the IC computing system to:
receive a message including admission data associated with at least one patient;
configure the admission data into input data for a machine learning (ML) model configured to automatically classify the data by admission type;
input the input data into the ML model;
based upon an output from the ML model, determine an admission type, of a plurality of admission types, associated with the at least one patient;
generate an authorization message associated with the at least one patient; and
transmit the authorization message to an external computing device for approval.
Embodiment 12. The non-transitory computer-readable storage medium of the preceding clause, wherein the message comprises an admission discharge transfer (ADT) message.
Embodiment 13. The non-transitory computer-readable storage medium of any preceding clause, wherein the plurality of admission types comprises at least one admission type selected from the group consisting of an obstetrics (OB) type, a behavioral health (BH) type, and a Medical type.
Embodiment 14. The non-transitory computer-readable storage medium of any preceding clause, wherein the ML model utilizes a factor selected from the group consisting of state, facility, age, sex, health plan, diagnosis (dx) code, visit duration, product, expected due date, inpatient future risk, risk score, inpatient stay probability, er risk score, nest score, ADT type, trimester 1 visit date, trimester 2 visit date, trimester 3 visit date, Clinical Classifications Software Refined, plan type, source, total BH risk score, er risk score fc, ip risk score, total risk score, and combinations thereof.
Embodiment 15. The non-transitory computer-readable storage medium of any preceding clause, wherein the ML model utilizes a factor selected from the group consisting of trimester 1 visit date, trimester 2 visit date, trimester 3 visit date, and combinations thereof.
Embodiment 16. The non-transitory computer-readable storage medium of any preceding clause, wherein the ML model utilizes a factor selected from the group consisting of age, facility, sex, inpatient future risk, cars, diagnosis code, health plan, adt type, and combinations thereof.
Embodiment 17. The non-transitory computer-readable storage medium of any preceding clause, wherein the ML model employs a neural network selected from the group consisting of a convolutional neural network, a deep learning neural network, a combined learning module, a program that learns in two or more fields or areas of interest, and combinations thereof.
Embodiment 18. A method implemented using an intelligent classification (IC) computing system including at least one database communicatively coupled to a processor, the method comprising:
receiving a message including admission data associated with at least one patient;
configuring the admission data into input data for a machine learning (ML) model configured to automatically classify the data by admission type;
inputting the input data into the ML model;
based upon an output from the ML model, determining an admission type, of a plurality of admission types, associated with the at least one patient;
generating an authorization message associated with the at least one patient; and
transmitting the authorization message to an external computing device for approval.
Embodiment 19. The method of the preceding clause, wherein the message comprises an admission discharge transfer (ADT) message.
Embodiment 20. The method of any preceding clause, wherein the plurality of admission types comprises at least one admission type selected from the group consisting of an obstetrics (OB) type, a behavioral health (BH) type, and a Medical type.
Embodiment 21. The method of any preceding clause, wherein the ML model employs a neural network selected from the group consisting of a convolutional neural network, a deep learning neural network, a combined learning module, a program that learns in two or more fields or areas of interest, and combinations thereof.
Embodiment 22. The method of any preceding clause, wherein the ML model is configured for pattern recognition and/or predictive modeling.

Having described aspects of the disclosure in detail, it will be apparent that modifications and variations are possible without departing from the scope of aspects of the disclosure as defined in the appended claims. As various changes could be made in the above constructions, products, and methods without departing from the scope of aspects of the disclosure, it is intended that all matter contained in the above description and shown in the accompanying drawings shall be interpreted as illustrative and not in a limiting sense.

While the disclosure has been described in terms of various specific embodiments, those skilled in the art will recognize that the disclosure can be practiced with modification within the spirit and scope of the claims.

As used herein, the term “database” may refer to either a body of data, a relational database management system (RDBMS), or to both. A database may include any collection of data including hierarchical databases, relational databases, flat file databases, object-relational databases, object oriented databases, and any other structured collection of records or data that is stored in a computer system. The above examples are for example only, and thus, are not intended to limit in any way the definition and/or meaning of the term database. Examples of RDBMS's include, but are not limited to including, Oracle® Database, MySQL, IBM® DB2, Microsoft® SQL Server, Sybase®, and PostgreSQL. However, any database implementation (e.g., relational, document-based) may be used that enables the system and methods described herein. (Oracle is a registered trademark of Oracle Corporation, Redwood Shores, California; IBM is a registered trademark of International Business Machines Corporation, Armonk, New York; Microsoft is a registered trademark of Microsoft Corporation, Redmond, Washington; and Sybase is a registered trademark of Sybase, Dublin, California).

The term processor, as used herein, may refer to central processing units, microprocessors, microcontrollers, reduced instruction set circuits (RISC), application specific integrated circuits (ASIC), logic circuits, and any other circuit or processor capable of executing the functions described herein.

As used herein, the terms “software” and “firmware” are interchangeable, and include any computer program stored in memory for execution by a processor, including RAM memory, ROM memory, EPROM memory, EEPROM memory, and non-volatile RAM (NVRAM) memory. The above memory types are for example only, and are thus not limiting as to the types of memory usable for storage of a computer program.

As used herein, the term “non-transitory computer-readable media” is intended to be representative of any tangible computer-based device implemented in any method or technology for short-term and long-term storage of information, such as, computer-readable instructions, computer-executable instructions, data structures, program modules and sub-modules, or other data in any device. Therefore, the methods described herein may be encoded as executable instructions embodied in a tangible, non-transitory, computer readable medium, including, without limitation, a storage device and/or a memory device. Such instructions, when executed by a processor, cause the processor to perform at least a portion of the methods described herein. Moreover, as used herein, the term “non-transitory computer-readable media” includes all tangible, computer-readable media, including, without limitation, non-transitory computer storage devices, including, without limitation, volatile and nonvolatile media, and removable and non-removable media such as a firmware, physical and virtual storage, CD-ROMs, DVDs, and any other digital source such as a network or the Internet, as well as yet to be developed digital means, with the sole exception being a transitory, propagating signal.

As used herein, the terms “comprises,” “comprising,” “includes,” “including,” “has,” “having,” “contains”, “containing,” “characterized by” or any other variation thereof, are intended to cover a non-exclusive inclusion, subject to any limitation explicitly indicated. For example, a composition, mixture, process or method that comprises a list of elements is not necessarily limited to only those elements but may include other elements not expressly listed or inherent to such composition, mixture, process or method.

The transitional phrase “consisting of” excludes any element, step, or ingredient not specified. If in the claim, such would close the claim to the inclusion of materials other than those recited except for impurities ordinarily associated therewith. When the phrase “consisting of” appears in a clause of the body of a claim, rather than immediately following the preamble, it limits only the element set forth in that clause; other elements are not excluded from the claim as a whole.

The transitional phrase “consisting essentially of” is used to define a composition or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

Where an invention or a portion thereof is defined with an open-ended term such as “comprising,” it should be readily understood that (unless otherwise stated) the description should be interpreted to also describe such an invention using the terms “consisting essentially of” or “consisting of.”

Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

As used herein, an element or step recited in the singular and preceded with the word “a” or “an” should be understood as not excluding plural elements or steps, unless such exclusion is explicitly recited. For example, reference to “an allocation plan” may refer to a plurality of allocation plans. Furthermore, references to “example embodiment” or “one embodiment” of the present disclosure are not intended to be interpreted as excluding the existence of additional embodiments that also incorporate the recited features.

As used herein, the term “about” means plus or minus 10% of the value.

As will be appreciated based on the foregoing specification, the above-described embodiments of the disclosure may be implemented using computer programming or engineering techniques including computer software, firmware, hardware or any combination or subset thereof. Any such resulting program, having computer-readable code means, may be embodied or provided within one or more computer-readable media, thereby making a computer program product, i.e., an article of manufacture, according to the discussed embodiments of the disclosure. The article of manufacture containing the computer code may be made and/or used by executing the code directly from one medium, by copying the code from one medium to another medium, or by transmitting the code over a network.

In addition, although various elements of IC computing device 102 are described herein as including general processing and memory devices, it should be understood that IC computing device 102 is a specialized computer configured to perform the steps described herein for automatic classification of data and generation of messages.

This written description uses examples to disclose the embodiments, including the best mode, and also to enable any person skilled in the art to practice the embodiments, including making and using any devices or systems and performing any incorporated methods. The patentable scope of the disclosure is defined by the claims, and may include other examples that occur to those skilled in the art. Such other examples are intended to be within the scope of the claims if they have structural elements that do not differ from the literal language of the claims, or if they include equivalent structural elements with insubstantial locational differences from the literal language of the claims.

What is claimed is:

1. An intelligent classification (IC) computing system comprising at least one processor in communication with at least one database, the at least one processor configured to:
receive a message including admission data associated with at least one patient;
configure the admission data into input data for a machine learning (ML) model configured to automatically classify the data by admission type;
input the input data into the ML model;
based upon an output from the ML model, determine an admission type, of a plurality of admission types, associated with the at least one patient;
generate an authorization message associated with the at least one patient; and
transmit the authorization message to an external computing device for approval.

2. The IC computing system of claim 1, wherein the message comprises an admission discharge transfer (ADT) message.

3. The IC computing system of claim 1, wherein the plurality of admission types comprises at least one admission type selected from the group consisting of an obstetrics (OB) type, a behavioral health (BH) type, and a Medical type.

4. The IC computing system of claim 1, wherein the ML model utilizes a factor selected from the group consisting of state, facility, age, sex, health plan, diagnosis (dx) code, visit duration, product, expected due date, inpatient future risk, risk score, inpatient stay probability, er risk score, nest score, ADT type, trimester 1 visit date, trimester 2 visit date, trimester 3 visit date, Clinical Classifications Software Refined, plan type, source, total BH risk score, er risk score fc, ip risk score, total risk score, and combinations thereof.

5. The IC computing system of claim 1, wherein the ML model utilizes a factor selected from the group consisting of trimester 1 visit date, trimester 2 visit date, trimester 3 visit date, and combinations thereof.

6. The IC computing system of claim 1, wherein the ML model utilizes a factor selected from the group consisting of age, facility, sex, inpatient future risk, cars, diagnosis code, health plan, adt type, and combinations thereof.

7. The IC computing system of claim 1, wherein the ML model employs a neural network selected from the group consisting of a convolutional neural network, a deep learning neural network, a combined learning module, a program that learns in two or more fields or areas of interest, and combinations thereof.

8. The IC computing system of claim 1, wherein the ML model is configured for pattern recognition and/or predictive modeling.

9. The IC computing system of claim 1, wherein the processor is further configured to produce a further output based on the automatic classification of the data.

10. The IC computing system of claim 1, wherein the processor is further configured to create a data file based on the automatic classification of the data.

11. A non-transitory computer-readable storage medium having computer-executable instructions embodied thereon, wherein when executed by an intelligent classification (IC) computing system including at least one processor in communication with at least one database, the computer-readable instructions cause the IC computing system to:
receive a message including admission data associated with at least one patient;
configure the admission data into input data for a machine learning (ML) model configured to automatically classify the data by admission type;
input the input data into the ML model;
based upon an output from the ML model, determine an admission type, of a plurality of admission types, associated with the at least one patient;
generate an authorization message associated with the at least one patient; and
transmit the authorization message to an external computing device for approval.

12. The non-transitory computer-readable storage medium of claim 11, wherein the message comprises an admission discharge transfer (ADT) message.

13. The non-transitory computer-readable storage medium of claim 11, wherein the plurality of admission types comprises at least one admission type selected from the group consisting of an obstetrics (OB) type, a behavioral health (BH) type, and a Medical type.

14. The non-transitory computer-readable storage medium of claim 11, wherein the ML model utilizes a factor selected from the group consisting of state, facility, age, sex, health plan, diagnosis (dx) code, visit duration, product, expected due date, inpatient future risk, risk score, inpatient stay probability, er risk score, nest score, ADT type, trimester 1 visit date, trimester 2 visit date, trimester 3 visit date, Clinical Classifications Software Refined, plan type, source, total BH risk score, er risk score fc, ip risk score, total risk score, and combinations thereof.

15. The non-transitory computer-readable storage medium of claim 11, wherein the ML model utilizes a factor selected from the group consisting of trimester 1 visit date, trimester 2 visit date, trimester 3 visit date, and combinations thereof.

16. The non-transitory computer-readable storage medium of claim 11, wherein the ML model utilizes a factor selected from the group consisting of age, facility, sex, inpatient future risk, cars, diagnosis code, health plan, adt type, and combinations thereof.

17. The non-transitory computer-readable storage medium of claim 11, wherein the ML model employs a neural network selected from the group consisting of a convolutional neural network, a deep learning neural network, a combined learning module, a program that learns in two or more fields or areas of interest, and combinations thereof.

18. A method implemented using an intelligent classification (IC) computing system including at least one database communicatively coupled to a processor, the method comprising:
receiving a message including admission data associated with at least one patient;
configuring the admission data into input data for a machine learning (ML) model configured to automatically classify the data by admission type;
inputting the input data into the ML model;
based upon an output from the ML model, determining an admission type, of a plurality of admission types, associated with the at least one patient;
generating an authorization message associated with the at least one patient; and
transmitting the authorization message to an external computing device for approval.

19. The method of claim 18, wherein the message comprises an admission discharge transfer (ADT) message.

20. The method of claim 18, wherein the plurality of admission types comprises at least one admission type selected from the group consisting of an obstetrics (OB) type, a behavioral health (BH) type, and a Medical type.
